(19) United States
(12) Patent Application Publication
(10) Pub. No.: US 2011/0212484 A1
(43) Pub. Date: Sep. 1, 2011

(54) In Vitro Model of Focal Ischemia

(76) Inventors: Marc J. P. Richard, Charlottetown (CA); Tarek M. Saleh, Stratford (CA); Bouchaib El Bahh, Charlottetown (CA); Jeffrey A. Zidichouski, Charlottetown (CA)

(21) Appl. No.: 13/125,380

(22) PCT Filed: Oct. 19, 2009
(86) PCT No.: PCT/CA2009/001500

§ 371 Date: Apr. 21, 2011

Related U.S. Application Data
(60) Provisional application No. 61/193,005, filed on Oct. 21, 2008.

(51) Int. Cl. C12Q 1/02

(52) U.S. Cl. 435/29

(57) ABSTRACT

A method of modeling in vitro focal ischemia comprising: perfusing a tissue slice in vitro with an oxygenated medium; and, applying a focal insult to a targeted portion of the tissue slice. The method is particularly useful for brain tissue.

### CROSS-REFERENCE TO RELATED APPLICATIONS

This application claims the benefit of U.S. Provisional Patent Application Ser. No. 61/193,005 filed Oct. 21, 2008, the entire contents of which his herein incorporated by reference.

### FIELD OF THE INVENTION

The present invention is related to models for simulating focal ischemia in a tissue, especially brain tissue, of an animal.

### BACKGROUND OF THE INVENTION

During a stroke, blood flow to an area of the brain is blocked (focal ischemic insult) and consequently that area of the brain begins to die. The initial area of neuronal death is called the core. As neurons within the core die, they release their cellular contents which migrate to neighbouring neurons and trigger processes that eventually lead to their death.

There is great interest in screening compounds for their ability to reduce the rate of spread of a focal ischemic insult. However, in order to efficiently screen such compounds, it is desirable to have a model that allows researchers to rapidly determine efficacy as well as to determine mechanism of action of the compounds. Such models would preferably provide a medium throughput while minimizing the number of animals required. Current models used in stroke research include: in vivo focal ischemia models, in vitro whole brain focal ischemia models, in vitro global ischemia brain slice models and in vitro dissociated cell models. None of the current models reproduce focal ischemia.

In vivo focal ischemia models (whole animal) are popular models for stroke research and involve invasive surgery to expose a cerebral artery and occlude it using electrocautery or injection of a preformed blot clot (model of permanent stroke). Transient stokes can be performed by inserting a suture into the carotid artery until it reaches the cerebral vasculature. The suture is left in place and then removed to simulate “reperfusion”. Following the simulated stroke (or reperfusion), cell death is quantified by using a stain (tetrazolium chloride; TTC) and measuring the area or volume of cell death. Although these models are the most physiologically accurate, they have a very low throughput, are technically demanding, time consuming and require the use of vast amounts of animals all which results in a very high cost per data point. As well, using this type of model, it is difficult to determine the mechanism of action of therapeutic interventions as well as to use as an effective drug screening tool or model.

An in vitro whole brain focal ischemia model has recently been designed in which the entire brain is removed and maintained under in vitro conditions. With this model the advantages of using an in vitro system is achieved in a brain that is physically and functionally intact, but it has the disadvantages associated with in vivo models with regard to throughput, cost, technical skills required and time required to conduct the experiments.

The use of an in vitro brain slice oxygen glucose deprivation (OGD) model overcomes many of the problems associated with whole brain or in vivo models. It is higher throughput and requires fewer animals than in vivo models. As well, it is an ideal model to use for physiological experiments to determine mechanism of action. One limitation of current in vitro slice models of stroke is that OGD media is applied to the entire brain slice and thus the entire slice undergoes an ischemic insult. Therefore, all of the cells in the slice are subject to the same ischemic condition (core) and is referred to as global ischemia.

In vitro dissociated cell models in which oxygen glucose deprivation (OGD) is by far the most common method used to date because it can be used in a medium to high-throughput fashion for drug screening. However, it is far removed from being physiologically accurate since the cells are dissociated through mechanical and enzymatic treatments and hence not at all similar to being in their normal environment. The major disadvantage of using dissociated cells is that they are usually pure cultures (only neurons with no glial cells) or cell lines derived from tumors (i.e. neuroblastomas, etc.) and these both are further removed from the normal physiological and neuroanataomical organization and function of these cells. Cell cultures also lack the synaptic contacts between neurons or between neurons and glial cells, which are important connections or associations that are severed during the dissociation procedure and allowed to “re-form” during incubation in culture. These reconnections take a number of days to reform and are not likely to be the same as they were in situ or in vivo.

Although stroke studies using in vivo models are the most physiologically accurate, they are low throughput, and therefore high cost experiments, and require the use of vast amounts of animals. As well, the ability to assess mechanism of action of drugs and to study neurophysiological network changes that occur with stroke is restricted in in vivo systems. Such studies are better suited for in vitro models. As described above, there is now an in vitro whole brain model available that incorporates some of the benefits of an in vitro model. However, the drawback of this model is that, like the in vivo model, it is low throughput and requires large numbers of animals because only one stroke can be performed per brain. Unfortunately, all current in vitro brain slice models mimic global ischemia rather than focal ischemia. Although these current models have been useful in characterizing ischemia-induced neuronal death and have utility in identifying compounds that are neuroprotective, or to demonstrate toxicity, they do not mimic the focalization of an actual stroke. Clinically speaking, the majority of strokes where the patient survives and embarks on the road to recovery are focal in nature (a cerebral artery, branch point or arteriole becomes occluded and damaged occurs only to the area(s) of the brain these vessels supply with oxygen and nutrients). Consequently, using these current global models, it is impossible to characterize the spread of cell death from the core to the adjacent healthy tissue.

There remains a need in the art for a reliable focal ischemic in vitro brain slice model.

### SUMMARY OF THE INVENTION

It has now possible to overcome the problem of global ischemia in tissue slices by focally applying an ischemic insult (e.g. oxygen glucose deprivation (OGD) medium) to a very small portion of a tissue slice in vitro while the remainder of the slice continues to be perfused with normal oxygenated media.

Thus, in one aspect of the present invention there is provided a method of modeling in vitro focal ischemia comprising: perfusing a tissue slice with an oxygenated medium; and, applying a focal insult to a targeted portion of the tissue slice.

The present model can also be used to mimic both focal and transient ischemic insults in a variety of tissues. Tissues include, for example, central nervous system tissue (e.g. brain tissue, spinal tissue, optic nerve), cardiac tissue, muscle tissue (e.g. skeletal muscle tissue), renal tissue, hepatic tissue and retinal tissue. Preferably, the present model is used in conjunction with slices of brain tissue or slices of cardiac tissue. Using slices of cardiac tissues permits mimicking of myocardial infarction (MI; heart attack). Use of the model in conjunction with slices of brain tissue is particularly preferred. Brain tissue includes, for example, tissue from the cortex, stem or cerebellum. Cortical tissue (i.e. tissue from the cortex of the brain) is particularly preferred.

Any suitable physiologically acceptable oxygenated medium may be used, for example, the oxygenated medium may comprise an oxygenated saline solution (e.g. artificial cerebro-spinal fluid (aCSF), Krebs solution or an equivalent cell culture medium such as Hank's, Eagles, MEM, etc.). Perfusion with oxygenated medium may be accomplished by any suitable method, for example, by bathing the tissue slice in the medium either by gravity fed or peristaltic pump systems. Preferably, the tissue slice is perfused with oxygenated medium before, during and after the ischemic insult is applied to the targeted portion. Tissue slices are preferably kept close to body temperature (e.g. 32-35° C.).

The focal insult may be caused by anything that can be focally applied to the tissue slice (e.g. brain slice) that causes cell death (e.g. neuronal cell death) in a small area of the slice. The focal insult may be caused by, for example, focal application of oxygen glucose deprivation (OGD) medium, focal application of a mechanical trauma or focal application of an acute chemical trauma.

Any suitable OGD medium may be used, for example, the OGD medium may comprise a deoxygenated saline solution. Other than being oxygen and glucose deprived, the OGD medium is preferably physiologically acceptable. Examples of OGD media are known (Konrath 2008; Rosa 2008). Application of OGD medium to the targeted portion of the tissue slice may be achieved by any suitable means, for example, with a micro-perfuser. The micro-perfuser may comprise, for example, a pump driven or gravity fed system. A pump driven system, e.g. using a peristaltic pump, is preferred. The micro-perfuser may further comprise a capillary tube with a very fine tip for precise perfusion of OGD to the targeted portion of the tissue slice. The capillary tube may comprise glass, stainless steel, any other suitable material, or combination thereof. Materials such as stainless steel are more permanent but materials such as glass are less expensive. Robotics, e.g. a micromanipulator, may be used to aid in accurate placement of the OGD medium. In order to maintain laminar flow of OGD medium onto a focal area, a flow rate in a range of from about 35-200 μL per minute, preferably 80-120 μL per minute, for example 100 μL per minute, may be used.

The targeted portion of the tissue slice is generally considerably smaller than the overall size of the tissue slice itself. In this way, a truly focal ischemic event can be mimicked. For example, in brain slices, preferably about 5-20% of the cortex of the brain slice is targeted. Preferably, the targeted portion is perfused with OGD medium (without reperfusion) for a period of time in a range of about 1-3 hours, more preferably about 2 hours.

Tissue slices may be obtained from any suitable test animal, for example, a rat, mouse, hamster, etc. Tissues are surgically removed from the animal and sliced using known methods. Tissue slices preferably have a thickness of no greater than about 400 μm, and are thin enough to be kept alive by external means in the absence of the normal vasculature.

In brains, the brain is preferably sliced in a manner that maintains synaptic contacts and glial cell composition. Brains may also be sliced in other ways, for example, a medullary-pontine slice which maintains fibre tracts and connections between the medullary and pontine regions, which are several millimeters apart. Further, the way in which brain slices are used enables multiple tests to be conducted on a single brain, as opposed to a whole animal model where one animal brain would be required for each test. Numerous hemisected cortical slices can be generated from a single brain, for example, about 40 preparations per rat, or about 30 preparations per mouse. Each hemisected slice may be further split into at least four pieces as the cortex is quite large. Thus, a team of two experimenters can do over 100 experiments per day, and perhaps over 300 experiments if the hemisected slices are further subdivided.

Studies using in vivo models are low throughput, require large numbers of animals and are limited in their ability to study neurophysiological changes and mechanism of action of drugs. Because the model of the present invention involves cutting the tissue into several slices, it is an advantage of the present invention that numerous tests can be performed from a single tissue thereby increasing throughput while reducing the number of animal used and consequently reducing the cost. Further, unlike in vitro dissociated cell models, the model of the present invention in respect of brains uses brain slices in which synaptic connections are still in tact, thereby mimicking in vivo conditions as closely as possible without the attendant difficulties of in vivo models.

Furthermore, unlike other in vitro tissue slice models, the present invention has been designed to mimic focal ischemia rather than global ischemia. Advantageously, this gives rise to several possible uses and/or applications for the present model which have hitherto been unrealized by in vitro tissue slice models.

One application of this model is to measure the change in infarct area (or volume) over time and to determine if target therapeutic or nutritional compounds can decrease or limit this infarct area (or volume). This is accomplished by staining for viable tissue following focal OGD application. This application could be used to study the effect of protective agents (e.g. neuroprotective agents), toxic agents (e.g. neurotoxic agent), physical trauma and/or pre-treatments on the size of the infarct area (or volume). Thus, it would be possible to determine whether potential (neuro)protective agents decrease infarct area (or volume), and whether potential (neuro)toxic agents increase infarct area (or volume). Test compounds could be applied acutely to the tissue slice, or the test animal could be pre-treated with the test compounds in advance. This is a marked improvement from current systems that generally use cell culture methods testing immortalized cells, for example immortalized CNS neurons (e.g. P-75), derived from a tumour source or using primary cultured cells that have issues with day to day consistency or have cryogenic-related susceptibilities. Thus, the present invention is useful in screening for therapeutic compounds and in determining mechanism of action of such compounds.

The model can also be used a trauma model where either focal mechanical trauma (e.g. crush) or acute chemical trauma that mimics a mechanically induced trauma and/or subsequent necrotic event (i.e., release of cell contents, glutamate, degradative enzymes, calcium) can be used to create a localized insult to the tissue that is under study. This model could therefore be used to examine for protection and/or prevention of cell damage in the affected area as well in areas adjacent a focal insult. It can also be used to rapidly assess neurotoxicity in a semi-intact nervous system, where damage can be assessed by focal application of a neurotoxic agent/insult delivered by focal application in a semi-intact nervous system. Further, by using a plurality of perfusions at disparate parts of a single tissue slice, a variety of new experiments may be more easily performed. For example, such a “multiplexing” method may be used for studying dose response relationships of protective or toxic agents in stroke or for studying the effect of multiple types of insults on the same tissue slice.

The present invention mimics the focal nature of the core seen in a stroke that occurs when a specific vessel is affected. Thus, in another application, the rate of spread of neuronal damage or cell death outwards from the core (i.e. spreading depression-wave of depolarization emanating out from the ischemic core) through otherwise healthy tissue with respect to time can be studied. This may be accomplished using electrophysiological techniques. Using these techniques, it is possible to measure, in real-time, changes in membrane potential and synaptic transmission at different distances from the ischemic core. It can then be determined whether or not different interventions can alter the time course or the amplitude of these changes. This cannot be done in any of the prior art in vitro systems mentioned above as neither synaptic contacts nor glial cell composition are maintained, both of which are important for spreading depression to be studied.

Advantageously, the present invention can also mimic the death and mechanism of cell death outside of the core region of the focal ischemia. The region outside of the core region is known as the penumbra, and contains cells susceptible to excitotoxicity due to glutamate release for the dead cells as well as due to synaptically released glutamate from dying neurons in the focal region. Because the present model can also mimic the penumbra, it is now possible to model the rescue of cells located in the penumbra region. Modeling the penumbra region in this manner can lead to better treatments for diminishing the size of the stroke's overall death zone (volume).

Previous cell-based focal ischemia models are unable to mimic both the core and penumbra regions in a suitable manner since cell-based assays cannot successfully replicate the physiologically conditions of cells, especially neuronal cells, as indicated previously. Since the present model employs tissues slices, the physiological conditions in which the cells exist is much closer to normal conditions permitting accurate modeling of both the core and penumbra regions of a focal ischemic event. This is a very significant advantage of the present model over prior art cell-based focal ischemia models, especially in relation to brain tissues.

There are other applications for the model of the present invention. Following a stroke, initial treatment is aimed at restoring blood flow to the ischemic region. Unfortunately, reperfusion in itself can be very damaging to tissue, especially neuronal tissue. This process is referred to as reperfusion injury and it is not completely understood. Using the present model, OGD solution can be focally applied to mimic a focal stroke, then, simply by shutting off the flow of OGD medium the ischemic tissue can be reperfused with oxygenated medium. It is also be possible to tissue culture the OGD treated tissue slices and thus be able to study the long term effects of reoxygenation. Thus, the present model can be very useful in studying reperfusion injury and in testing compounds aimed at reducing this injury.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIGS. 1A-1E are digital images of brain slices focally perfused with various solutions;

FIG. 2A is a schematic diagram depicting a brain slice and showing locations of stimulating and recording electrodes relative to a region of focally applied OGD solution in an experiment to follow progression of an ischemic event in the brain slice;

FIGS. 2B-2C depict representative electrophysiological recordings of field potentials in a region adjacent to the focal OGD application in the experiment depicted in FIG. 2A;

FIGS. 3A-3B depict representative recordings of membrane potential of cortical neurons in the core region of an anoxic depolarization (AD) event comparing the effect of different amounts of edaravone on membrane potential recovery;

FIGS. 3C-3D depict mean data from multiple recordings of membrane potential demonstrating the effect of different amounts of edaravone on membrane potential recovery;

FIGS. 3E-F depict mean data demonstrating the effects of edaravone on the latency of anoxic depolarization (AD) in a focal ischemic event;

FIGS. 4A-B depict representative recordings of membrane potential comparing the effect of OGD and glutamate on cell death;

FIG. 5A is a representative recording of membrane potential in the penumbra region of a focal ischemic event;

FIG. 5B depicts mean data of multiple recordings of membrane potential in the penumbra region of a focal ischemic event;

FIG. 5C depicts a graph illustrating percentage neuronal survival in the penumbra region of a focal ischemic event with and without the addition of edaravone;

FIG. 6A depicts a diagram showing placement of recording electrodes in relation to a focal glutamate perfusion of a brain slice in an experiment to determine effect of focal glutamate perfusion at three different distances from the perfusion site;

FIGS. 6B-D depict representative recording of membrane potential taken from cortical neurons located at the three different distances indicated in FIG. 6A;

FIG. 7A depicts a digital image of a rat liver slice focally perfused with deoxygenated, glucose-free Krebs solution illustrating the utility of the present invention for modeling focal ischemia in hepatic tissue; and,

FIG. 7B depicts a digital image of a rat heart slice focally perfused with deoxygenated, glucose-free Krebs solution illustrating the utility of the present invention for modeling focal ischemia in cardiac tissue.

### DESCRIPTION OF PREFERRED EMBODIMENTS

### Materials and Methods:

All experiments were carried out in accordance with the guidelines of the Canadian Council on Animal Care and were approved by the University of Prince Edward Island Animal Care Committee.

### Brain Slice Preparation

In vitro focal ischemia studies were performed on freshly prepared rat brain slices.

Methods for preparing the brain slices were similar to those previously published (Fatehi et al 2006). Briefly, male Sprague-Dawley rats (100-125 g; Charles River, Montreal, PQ, Canada) were anaesthetized with isoflurane vapour (Isoflo™; Abbott Laboratories, Saint-Laurent, PQ) and then decapitated. Brains were removed and immersed in ice-cold (2-3° C.) artificial cerebrospinal fluid (aCSF) of the following composition (in mM): 145 NaCl, 2.5 KCl, 10 D-glucose, 26 NaHCO₃, 1.2 NaH₂PO₄, 1.3 MgCl₂, 2.5 CaCl₂ (pH 7.4, osmolarity of 295-305 mOsmol/L, continuously bubbled with 95% O₂ and 5% CO₂). The brain was then mounted in a vibratome (VT 1000S, Leica) and cut coronally into 400 μm thick slices while submersed in ice-cold aCSF. Prior to the initiation of experiments, slices were incubated for at least 1 hour in aCSF at room temperature.

### Brain Slice Experimental Conditions

For experimentation, slices were transferred to an experimental chamber on an upright microscope and superfused at 3 ml/min with aCSF bubbled with 95% O₂ and 5% CO₂. The aCSF was gravity fed into the chamber and was heated to 34° C.±1° C. with an in-line solution heater (SHM-6, Warner Instruments, Hamden, Conn., USA) connected to a heater controller (TC-344B, Warner Instruments, Hamden, Conn., USA). Solution was removed from the opposite end of the chamber via a suction tube. In order to facilitate good fluid circulation over both sides of the slice, the slice was suspended on a mesh insert placed within the bath. The slice was secured in place by a slice anchor hold-down (thin wire loop with fine thread spaced approximately 1 mm apart). The slices were allowed to equilibrate in the heated chamber for approximately 30 minutes prior to the commencement of experimentation.

### Heart and Liver Slice Preparation

Heart and liver slices were prepared and treated similar to brain slices with the exception that Krebs solution was used in place of aCSF solution. The composition of the Krebs solution was as follows: 118 mM NaCl; 4.7 mM KCl; 11.1 mM glucose; 1.2 mM NaH₂PO₄; 1.2 mM MgSO₄; 25 mM NaHCO₃; and, 2.5 mM CaCl₂ (continuously bubbled with 95% O₂ and 5% CO₂). Heart slices were prepared from the left ventricle. For the Krebs-OGD solution, glucose was substituted with D-mannitol and the solution was bubbled with 95% N₂ and 5% CO₂.

### Focal OGD Application

In order to mimic in vivo focal ischemia in the present in vitro system, oxygen-glucose deprived (OGD) medium was focally applied to a small area (0.25-0.5 mm wide or 5-10% of cortex) within the cortex of a brain slice, or within a slice of liver or heart tissue. The composition of this OGD medium for brain slices was as follows (in mM): 145 NaCl, 2.5 KCl, 26 NaHCO₃, 1.2 NaH₂PO₄, 1.3 MgCl₂, 2.5 CaCl₂, 10 D-mannitol (pH 7.4, osmolarity of 295-305 mOsmol/L). The OGD medium for liver and heart slices was deoxygenated, glucose-free Krebs solution. Oxygen was displaced from the media by bubbling with 95% N₂ and 5% CO₂. Focal application of OGD media was achieved with a microperfusion system driven by a syringe infusion pump (Physio 22 pump, Harvard Apparatus, Holliston, Mass., USA). Nozzle of the microperfusion system was made from a 23 gauge stainless steel tube and was mounted to a micromanipulator in order to ensure accurate placement. Using the micromanipulator the nozzle was lowered until it was just touching the surface of the slice. A heating element connected to a heater controller (TC-344B, Warner Instruments, Hamden, Conn., USA) was attached near the end of the nozzle in order to warm the focally applied media (34° C.±1° C.). The rate of focal solution application can be varied by adjusting the speed of the pump, however, the optimal OGD application rate was found to be about 100 μL per minute.

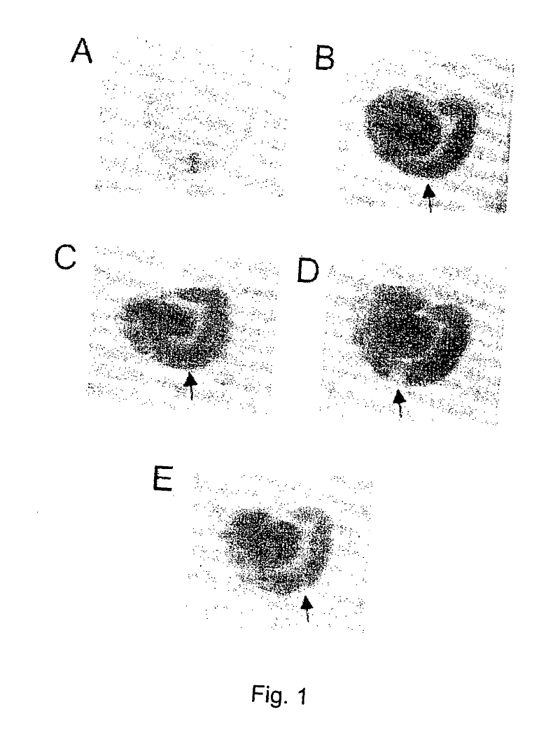

In order to minimize mixing of the OGD media with the surrounding aCSF media, the flow of solution within the bath was laminar. Laminar flow as well as the area of the focal solution application were confirmed by pumping dye (Chicago sky blue, Sigma) through the focal perfusion system (FIG. 1A). Once focal OGD application was established, different types of experiments were performed. These experiments are described below.

### Example 1

### Screening for Therapeutic Compounds

Referring to FIG. 1, rat brain slices were bath perfused with oxygenated aCSF solution. Different solutions were then focally applied to these slices at 100 μL/min. Arrows indicate the location of focal solution application. In FIG. 1A, Chicago sky blue tissue stain was focally applied to the slice. The stained tissue (dark area in FIG. 1A) represents the area of the focal solution application.

Following focal solution application, slices were incubated in a 2% solution of 2,3,5-triphenol tetrazolium chloride (TTC; Sigma-Aldrich; St. Louis; MO, USA) for 10 minutes at 37° C. With this stain, healthy tissue appeared red (dark areas in FIGS. 1B-1E) while dead tissue appeared white (light areas in FIGS. 1B-1E). Immediately following staining, the slices were placed in 10% fomalin in order to arrest the staining and preserve the slice. The slice was then digitally imaged using a flatbed scanner and quantification of infarct area was accomplished using a computer-assisted imaging system (ImageJ™, National Institutes of Health). In FIG. 1B, oxygenated aCSF solution was focally applied to the slice for 2 hours in order to ensure that focal solution application was not damaging the tissue. In FIG. 1C, OGD solution was focally applied to the slice for 1 hour. A white band (light area near arrow) on the cortex indicates cell death caused by focal ischemia. In FIG. 1D, OGD solution was focally applied to the slice for 2 hours. Compared to the 1 hour OGD application (FIG. 1C) the area of cell death is increased. In FIG. 1E, 100 μM lidocaine was added to the OGD media and this solution was focally applied to the slice for 2 hours. Lidocaine reduced the area of cell death caused by OGD.

### Example 2

### Progression of Focal Ischemia Event

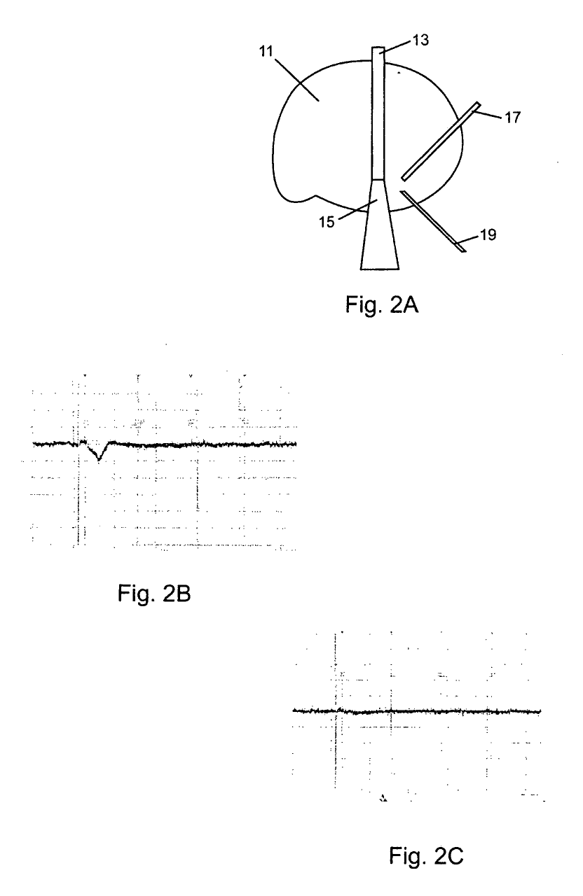

Referring to FIG. 2A, OGD solution was focally applied to the cortex of rat brain slice 11 from microperfusion nozzle 13 of a microperfusion system. Field excitatory postsynaptic potentials (fEPSP) were recorded from cortical tissue adjacent to region of focal OGD application 15. Locations of stimulating electrode 17 and recording electrode 19 relative to the region of focally applied OGD solution 15 are shown in FIG. 2A. FIGS. 2B-2C depict representative electrophysiological recordings of field potentials in the region adjacent to the focal OGD application. At the beginning of the experiment a fEPSP is present (FIG. 2B), however, after only 6 minutes of focal ODG application the fEPSP is lost (FIG. 2C).

### Example 3

### Mimicking the “Core” Region of Focal Ischemia

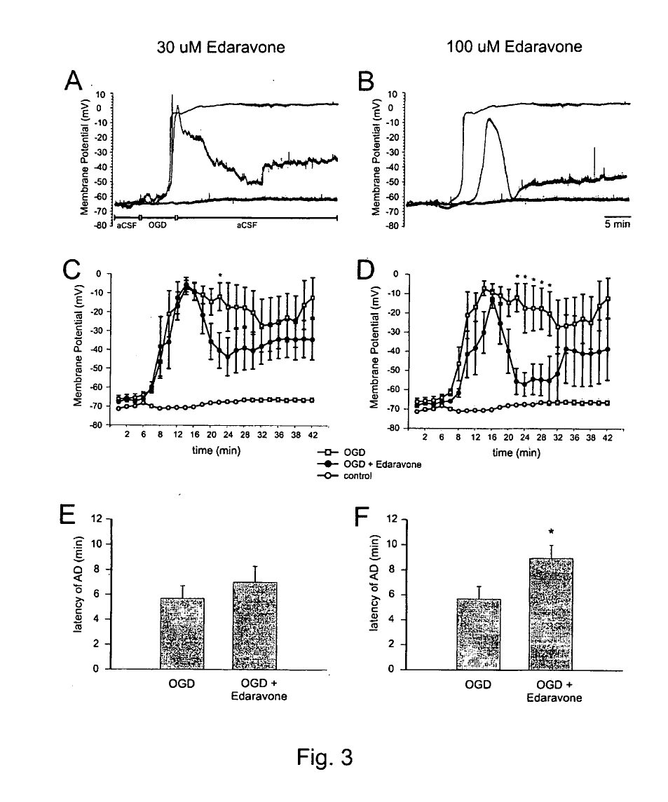

Rat brain slices were bath perfused with oxygenated aCSF solution and different solutions were focally perfused to these slices at 100 μL/min. aCSF solution was focally perfused for the first 5 minutes then OGD solution was focally perfused until the neuron depolarized. The solution was then switched back to aCSF for the remainder of the experiment. Intracellular recordings of membrane potential were obtained from cortical neurons within the region of the focal perfusion. Representative recordings of membrane potential are depicted in FIG. 3A and FIG. 3B. When neurons were continuously perfused with aCSF solution, membrane potential remained constant for the duration of the experiment. In contrast, perfusion of neurons with OGD solution caused a rapid depolarization of the neurons (anoxic depolarization (AD)). The addition of either 30 μM edaravone (FIG. 3A) or 100 μM edaravone (FIG. 3B) enhanced the recovery of membrane potential following AD, and 100 μM edaravone delayed the onset of AD.

FIG. 3C and FIG. 3D provide mean data demonstrating that 30 μM (FIG. 3C) and 100 μM (FIG. 3D) edaravone both significantly increased repolarization (recovery) of the neurons following AD caused by OGD. FIG. 3E and FIG. 3F provide mean data demonstrating the effects of edaravone on the latency of AD. 100 μM edaravone (FIG. 3F), but not 30 μM edaravone (FIG. 3E), significantly increased the time to onset of the AD. These data indicate that the this model can mimic conditions of the “core” region of focal ischemia and can effectively be used to test compounds targeted at reducing damage within this core region. In FIGS. 3C-F the * denotes p<0.05.

### Example 4

### Demonstration that Focal OGD and Glutamate Induced Cell Deaths are Similar

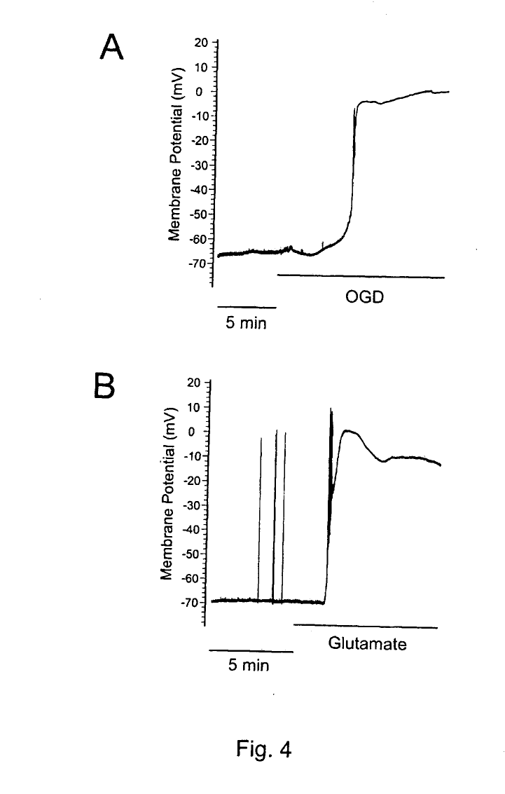

Rat brain slices were bath perfused with oxygenated aCSF solution. Either OGD solution or aCSF solution containing 20 mM glutamate was focally applied to these slices at 100 μL/min. Intracellular recordings of membrane potential were obtained from cortical neurons within the region of the focal perfusion. Representative recordings are depicted in FIG. 4A and FIG. 4B and demonstrate that both OGD (FIG. 4A) and glutamate (FIG. 4B) caused a rapid depolarization of the neuron. This suggests that the mechanism of OGD mediated cell death is likely due to glutamate induced excitotoxicity.

### Example 5

### Mimicking the “Penumbra” Region of Focal Ischemia

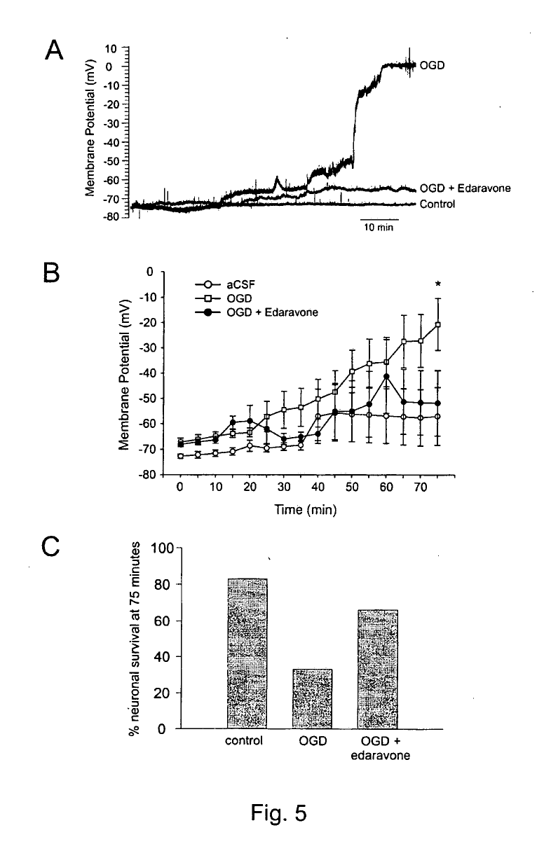

OGD solution was focally perfused onto rat brain slices bathed in oxygenated aCSF solution. Intracellular recordings of membrane potential were obtained from cortical neurons adjacent to the region of this focal OGD perfusion (approximately 0.9 mm from focal perfusion). A representative recording of membrane potential is depicted in FIG. 5A. Focal perfusion of OGD caused neurons outside the region of perfusion to progressively depolarize. When edaravone (100 μM) was added to the OGD media this depolarization was attenuated. This is reflected in the mean data (FIG. 5B). Percentage neuronal survival after 75 minutes is reflected in FIG. 5C. Neurons were considered “dead” if they became irreversibly depolarized to approximately 0 mV. The addition of 100 μM edaravone doubled neuronal survival from OGD. These data indicate that this model can be used to record neuronal changes in regions away from the core and is thus a model of ischemic penumbra. In FIG. 5 the * denotes p<0.05.

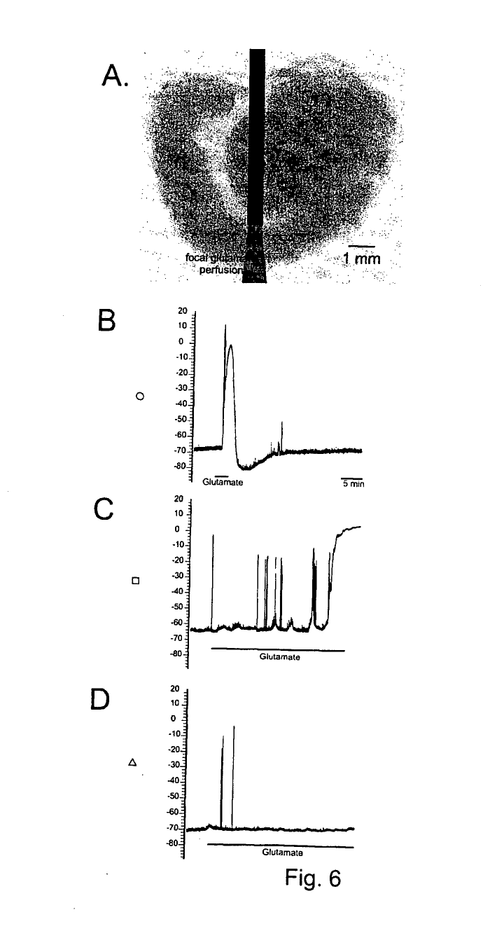

Using the present model, an experiment was also conducted to determine the effect of focal glutamate perfusion at different distances from the focal perfusion. Rat brain slices were bath perfused with oxygenated aCSF solution, and then aCSF solution containing 20 mM glutamate was focally perfused over the slice. Intracellular recordings of membrane potential were recorded from cortical neurons located at varying distances from the region of the focal perfusion. FIG. 6A depicts a diagram showing the placement of the recording electrodes in relation to the focal glutamate perfusion (∘=0.6 mm, □=0.9 mm, Δ=1.2 mm). FIG. 6B, FIG. 6C and FIG. 6D depict representative recordings of membrane potential taken from cortical neurons located at the varying distances from the focal glutamate perfusion. Referring to FIG. 6B, the neuron located 0.6 mm from the focal perfusion depolarized within a few minutes. Referring to FIG. 6C, this depolarization was delayed in the neuron located 0.9 mm from the focal perfusion. Referring to FIG. 6D, this depolarization was absent from the neuron located 1.2 mm from the focal perfusion. It is evident that the effect of focal glutamate perfusion is proportional to the distance from the focal perfusion. Thus, this indicates that the response in the penumbra is dependant on the events spreading from the core.

### Example 6

### Application to Focal Ischemia in Hepatic and Cardiac Tissue

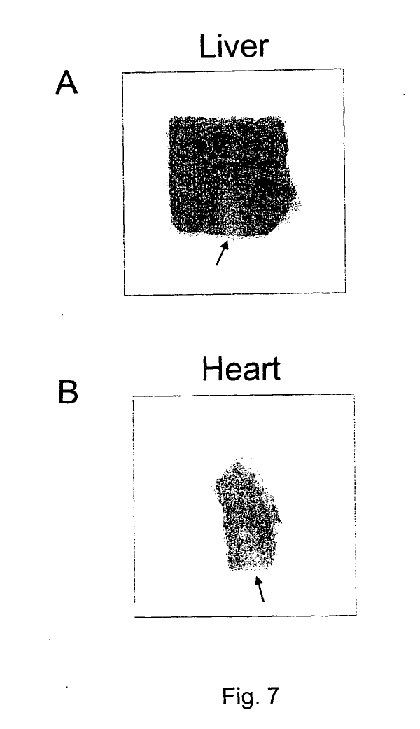

The present model can be used to produce focal insults in tissue other than the brain. Referring to FIG. 7, slices of rat liver (FIG. 7A) and a rat heart (FIG. 7B) were bath perfused with Krebs solution bubbled with 95% O₂/5% CO₂ (35° C.). A deoxygenated, glucose-free Krebs solution (bubbled with 95% N₂/5% CO₂) was focally perfused over a portion of the slices at 100 μL/min for 3 hours. At the end of the protocol, slices were incubated in 2% TTC stain (Sigma-Aldrich; St. Louis; MO, USA) for 10 minutes. With this stain, healthy tissue appears red while dead tissue appears white. The arrows in FIG. 7 indicate the locations of the focal solution applications. The white bands on the tissues are indicative of cell death caused by the focal ischemia.

### REFERENCES

The contents of the entirety of each of which are incorporated by this reference.
Fatehi M, Zidichouski J A, Kombian S B, Saleh T M (2006) Journal of Neuroscience Research. 84:666-674.
Hochman D W (1999) U.S. Pat. No. 5,976,825 issued Nov. 2, 1999.
Konrath L E, Santin K, Nassif M, Latini A, Henriques A, Salbego C (2008) J. Neuroscience doi:10.1016/j.neuro.2008.05.008.
Rosa et al. (2008) Exp Neurol. 212:93-99.
Wieloch T, Rytter A, Cronberg T (2005) U.S. Pat. No. 6,846,641 issued Jan. 25, 2005.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of modeling in vitro focal ischemia comprising: perfusing a tissue slice with an oxygenated medium in vitro; and, applying a focal insult to a targeted portion of the tissue slice.

2. The method according to claim 1, wherein the tissue comprises central nervous system tissue, cardiac tissue, muscle tissue, renal tissue, hepatic tissue or retinal tissue.

3. The method according to claim 1, wherein the tissue comprises brain tissue.

4. The method according to claim 1, wherein the tissue comprises cardiac tissue or hepatic tissue.

5. The method according to claim 1, wherein the focal insult comprises focal application of oxygen glucose deprivation (OGD) medium, focal application of a mechanical trauma or focal application of an acute chemical trauma.

6. The method according to claim 1, wherein the focal insult comprises focal application of oxygen glucose deprivation (OGD) medium.

7. The method according to claim 6, wherein the OGD is applied by microperfusion.

8. The method according to claim 7, wherein the microperfusion is accomplished with a microperfuser comprising a capillary tube and a pump for pumping the OGD through the capillary tube.

9. The method according to claim 6, wherein the OGD medium comprises a deoxygenated saline solution.

10. The method according to claim 1, wherein the targeted portion of the tissue slice is in a cortex of a brain slice.

11. The method according to claim 10, wherein the targeted portion of the brain slice has a size that is 5-20% of the cortex.

12. The method according to claim 1, wherein the tissue slice is perfused with oxygenated medium before, during and after the ischemic insult is applied to the targeted portion.

13. The method according to claim 1, wherein the perfusion with oxygenated medium is done by bathing the tissue slice in the oxygenated medium.

14. The method according to claim 1, wherein the oxygenated medium comprises an oxygenated saline solution.

15. The method according to claim 1, wherein the oxygenated medium comprises artificial cerebro-spinal fluid and the tissue slice is a brain slice.

16. The method according to claim 1, wherein the tissue slice has a maximum thickness of 400 μm.

17. Use of the method as defined in claim 6 for screening effect of test compounds on a focal ischemic event.

18. Use of the method as defined in claim 1 for following progression of a focal ischemic event.
